(19) United States
(12) Patent Application Publication
(10) Pub. No.: US 2013/0298637 A1
(43) Pub. Date: Nov. 14, 2013

(54) ANGLE CONNECTOR FOR CONNECTING A FLUID SENSOR TO A FLUID LINE

(71) Applicant: VERITAS AG, Gelnhausen (DE)

(72) Inventors: Elmar Sachs, Jossgrund (DE); Oliver Wilhelmi, Linsengericht (DE)

(73) Assignee: Veritas AG, Gelnhausen (DE)

(21) Appl. No.: 13/889,468

(22) Filed: May 8, 2013

(30) Foreign Application Priority Data
May 11, 2012 (DE) 10 2012 207 892.4

(51) Int. Cl. G01N 33/00; F16L 21/00

(52) U.S. Cl. CPC G01N 33/0004; F16L 21/00
USPC 73/23.2; 285/179

(57) ABSTRACT

An angle connector for the connection of a fluid sensor connecting piece of a fluid sensor to a fluid line, with a line connection for connecting the fluid line and a connecting sleeve for holding of the fluid sensor connecting piece with positive locking.

### BACKGROUND OF THE INVENTION

### Field of the Invention

The invention concerns an angle connector for the connection of a fluid sensor connecting piece, as well as a fluid sensor system to an angle connector.

In systems with flowing fluids, such as, for example, the exhaust gases in a vehicle internal combustion engine, the determination of a fluid property, for example, of a fluid pressure or of a fluid composition, is of great importance. However, for this purpose it is necessary to connect a fluid sensor to a line that guides the fluid. This can be especially problematic in vehicles, because existing connecting options for the connection of a fluid sensor connecting piece within a vehicle are often bulky and, based on an existing fuel line arrangement, difficult to mount and use.

The publication DE10 2009 039 983 A1 concerns a connecting element for flowing fluids consisting of a connector part with at least two connecting sections, each for connecting to a fluid line. However, this connecting element does not provide a compact possibility of connection for a fluid sensor.

### SUMMARY OF THE INVENTION

The invention provides a compact possibility of connection for a fluid sensor.

Accordingly, the invention provides an angle connector for the connection of a fluid sensor connecting piece of a fluid sensor to a fluid line with a line connection for connecting of the fluid line and a connecting sleeve for holding of the fluid sensor connecting piece with positive locking. The invention also provides a fluid sensor system, with an angle connector of the invention, a fluid sensor with a fluid sensor connecting piece, which is arranged in the connective sleeve and is connected to the connecting sleeve with positive locking, and a fluid line, which is connected to the line connection.

The invention is based on the recognition that a compact possibility of connection for a fluid sensor can be achieved by an angled connector that connects a sensor element, for example a sensor connecting piece, directly to a fluid-guiding line.

According to one aspect, the invention is concerned with an angle connector for the connection of a fluid sensor connecting piece of a fluid sensor to a fluid line with a line connection for the connection of the fluid line and a connecting sleeve for the positive locking of the fluid sensor connecting piece. As a result of this, for example, the technical advantages are achieved that the angle connector can be used with little structural space in the piping layouts, that nearly material-independent connection possibilities exist and that a sealing effect can be achieved with widely varied geometry. The fluid can be a gas or a liquid.

In an advantageous embodiment of the angle connector, a longitudinal axis of the line connection and a longitudinal axis of the connecting sleeve can be directed at an angle to one another. As a result of this, for example, the technical advantage is achieved that the angle connector has an especially compact form.

In a further advantageous embodiment of the angle connector, the angle between the longitudinal axis of the line connection and the longitudinal axis of the connecting sleeve lies in an angle range of between 80° and 100°. As a result of this, for example, the technical advantage is achieved that the fluid line can be connected to the fluid sensor from the side.

In a further advantageous embodiment of the angle connector, the line connection and the connecting sleeve each have an inner fluid channel that are connected to one another with fluid technology. As a result of this, for example, the technical advantage is achieved that the fluid sensor can measure a property of the fluid inside the angle connector and that the construction of the angle connector is kept compact.

In a further advantageous embodiment of the angle connector, the line connection and the connecting sleeve are made in one piece from a form-stable material. As a result of this, the technical advantage is achieved that the stability and the breaking strength of the angle connector are increased.

In a further advantageous embodiment of the angle connector, the connecting sleeve is designed to hold the fluid sensor connecting piece with the aid of a positively locking connecting piece. As a result of this, the technical advantage is achieved that mounting of the angle connector is simplified.

In a further advantageous embodiment of the angle connector, an inner wall of the connecting sleeve has at least one boss or recess for the holding of the fluid sensor connecting piece with positive locking. As a result of this, the technical advantage is achieved that the tightness of the connection is increased.

In a further advantageous embodiment of the angle connector, a molded part is arranged in the connecting sleeve for the holding of the fluid sensor connecting piece with positive locking. As a result of this, the technical advantage is achieved that the tightness of the connection is increased.

In a further advantageous embodiment of the angle connector, the molded part has a boss or a recess for the holding of the fluid sensor connecting piece with positive locking. As a result of this, the technical advantage is achieved that unintended loosening of the angle connector is prevented.

In a further advantageous embodiment of the angle connector, the molded part can be removable or permanently attached to a sleeve inner wall. As a result of this, the technical advantage is achieved that both the tensile strength as well as the tightness of the connection is increased.

In a further advantageous embodiment of the angle connector, the molded part is made from an elastic plastic, especially from an elastomer plastic. As a result of this, the technical advantage is achieved that an especially positive-locking connection is created and the tightness is improved still further.

In a further advantageous embodiment of the angle connector, the line connection has at least one retaining rib to hold the fluid line. As a result of this, the technical advantage is achieved that sliding down of the fluid line from the angle connector is prevented.

In a further advantageous embodiment of the angle connector, the line connection can be introduced into an inner fluid channel of the fluid line. As a result of this, the technical advantage is achieved that the connection of the line is protected from damage.

In a further advantageous embodiment of the angle connector, the connecting sleeve comprises a sealing ring. As a result of this, for example, the technical advantage is achieved that the tightness of the connection can be increased with simple means.

In a further advantageous embodiment of the angle connector, the molded part comprises at least two circumferential ribs for sealing the molded part against the connecting sleeve. As a result of this, for example, the technical advantage is achieved that improved sealing function is achieved.

According to a further aspect, the task according to the invention is achieved by a fluid sensor system with an angle connector according to the embodiments described above, a fluid sensor with a fluid sensor connecting piece that is arranged in the connecting sleeve and is joined to the connecting sleeve with positive locking and a fluid line that is connected with the line connection. As a result of this, the same advantages are achieved as by the angle connector described above.

### BRIEF DESCRIPTION OF THE DRAWINGS

Practical examples of the invention are shown in the drawing and will be described in more detail below.

The following are shown:

FIG. 1 is a cross-sectional view through a first embodiment of an angle connector;

FIG. 2 is a cross-sectional view through a second embodiment of an angle connector; and

FIG. 3 is a perspective view of a fluid sensor system.

### DETAILED DESCRIPTION

Many different sensors are used in vehicles of modern construction. Some of these sensors serve as fluid sensors for measuring the properties of a fluid, for example a gas or a liquid. Such a fluid sensor, for example, can determine a temperature, a pressure, a density, a composition, a flow velocity, a moisture content or another physical property of the fluid to be investigated. For this purpose, the fluid sensor usually has a fluid connection which, for example, can be a measuring head or a coupling piece for the guidance of the fluid further to a measuring head.

The fluid is guided in a fluid line. This fluid line is, for example, fiber-reinforced rubber tubing inside of which the fluid is transported. In a different embodiment, the fluid line is made of a plastic tube. In general, any device can be used as fluid line that is suitable for guiding or transporting a fluid inside it.

In order to measure the desired property of the fluid, the fluid sensor is brought into contact with the fluid. For this purpose, the fluid sensor is connected to the fluid line. Conventional connectors for this task are bulky and poorly suited to the spatial conditions inside a vehicle. The angle connector according to the invention solves this problem and makes reduction of the necessary constructional space possible while, at the same time, maintaining a complex line structure.

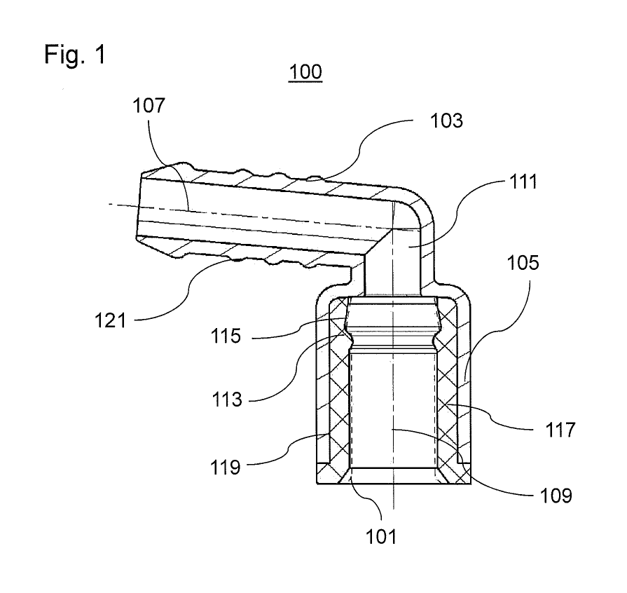

FIG. 1 shows a cross-sectional view through a first embodiment of an angle connector 100. The angle connector 100 comprises a line connection 103 for the connection of a fluid line and a connecting sleeve 105 for holding with positive locking of the fluid sensor connecting piece 101 that is shown with dotted lines. The fluid sensor connecting piece 101 can be introduced into the connecting sleeve 105 and the angle connector 100 is attached to the fluid sensor connector 101 with the connecting sleeve 105.

The connecting sleeve 105 is constructed with rotational symmetry around an axis 109. The line connection 103 serves for connecting and joining to a fluid line which guides the fluid or the fluid to the fluid sensor connecting piece 101. For example, the line connection 103 can be designed in such a way that a tube, as a fluid line, can be attached to the line connection. The line connection 103 is constructed symmetrically around the axis 107 and has a retaining rib 121 that, for example, prevents the tube from slipping off. The fluid is guided in the angle connector in an inner fluid channel 111.

The fluid sensor is arranged, for example, in an exhaust gas system, an aeration system, a fuel system, an oil or lubricant system, a coolant system, a brake system or any other system in which a fluid is used. A molded part 117 is arranged inside the connecting sleeve 105, made, for example, from an elastic material. The connecting sleeve 105 and the molded part 117 are, for example, solidly attached to one another on a contact surface area, for example glued together.

A circumferential boss 113 or a circumferential recess 115 is formed on the inner wall of the sleeve 119, which engages into a corresponding form in the fluid sensor connecting piece 101, and thereby improves the sealing effect of the angle connector 100. Moreover, as a result of this measure it is achieved that unintended loosening of the mechanical connection between the fluid sensor connecting piece 101 and the connecting sleeve 105 is prevented. In a further embodiment, the connecting sleeve comprises a sealing ring. The sealing wall of the molded part 117 can be adjusted to any contour of the fluid sensor connecting piece. By back-cutting, a connection can be made pressure-tight, for example, up to 6 bar.

In a further embodiment of the angle connector 100, the molded part 117 lying on the connecting sleeve 105 can be omitted. In this case the connecting sleeve 105 can have a positively locking structure.

The angle connector 100, the line connection 103, the connecting sleeve 105 or the molded part 117 can be made of a gas-tight material, for example a plastic. Suitable plastics are, for example, polyamide, polybutadiene, polyvinyl chloride, polypropylene, or polyethylene.

In an alternative embodiment the angle connector 100, the line connection 103, the connecting sleeve 105 or the molded part 117 can be made, for example, from fluorinated rubber material (FKM material). The fluorinated rubber material designates an entire group of rubbers which, as a common characteristic, all have vinylidene (di)fluoride (VDF) as one of their monomers. In a special embodiment, the fluorinated rubber material is a fluoroelastomer cross-linked with peroxide, which is filled with soot and which has a fluorine content of approx. 68%. This material is especially suitable because it has outstanding flexibility in the cold and has a melting point that lies at approx. −20° C.

In general, the manufacturing material is not limited to a specific material, and different materials can be used for the manufacture of the individual components. In general, it is advantageous to use different materials for the individual components and to combine their advantageous properties. The material of the connecting sleeve of the angle connector can be chosen arbitrarily due to the mechanically occurring sealing action.

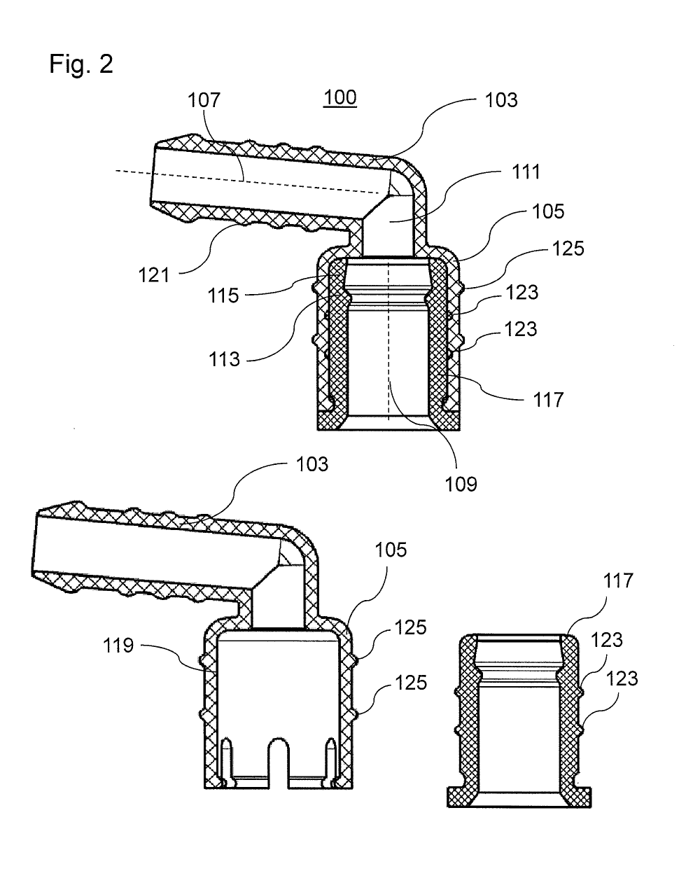

FIG. 2 shows a cross-sectional view through a second embodiment of an angle connector 100. In this embodiment, the connecting sleeve 105 is locked to the molded part 117. The molded part 117 comprises two circumferential ribs 123. The ribs 123 protrude on the outside of the molded part 117 and run parallel to one another around the molded part 117, which has a round cross-section. When the fluid sensor connector is in use, the circumferential ribs 123 increase the pressure of the molded part 117 on the smooth sleeve inner wall 119 and act as an additional sealing element. Thus, improved sealing function is achieved by the circumferential ribs 123 on the outside of the molded part 117.

In addition, the connecting sleeve 105 has on the outside two parallel circumferential reinforcing ribs 125, which strengthen the structure of the connecting sleeve 105 and facilitate the engagement of the connecting sleeve 105, for example when the angle connector 100 is removed. The other reference numbers designate the same components as those in FIG. 1.

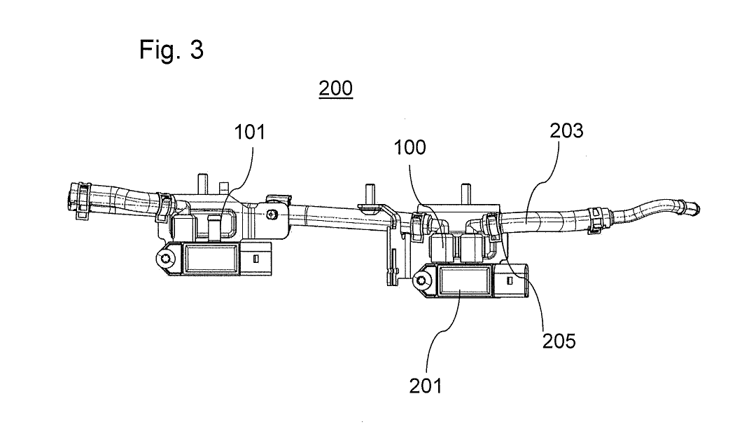

FIG. 3 shows a perspective view of a fluid sensor system 200. The fluid sensor system comprises the fluid sensors 201 and the fluid lines 203, such as, for example, rubber tubing. The fluid lines 203 and fluid sensors 201 are connected to one another with the aid of the angle connector 100. For this purpose, the connecting sleeve 105 is attached to the fluid sensor connecting piece 101. The fluid lines 203 are attached to the line connection 103 and are secured with clamps 205.

Through the angle connector 100 according to the invention, the necessary constructional space in the piping layout is reduced and a material-independent sealing action is achieved. By adjustment of the connecting sleeve or molded part inside the connecting sleeve, a sealing action is achieved that is independent of the geometry of the fluid sensor connecting piece. An angle connector with rotationally symmetrical connecting sleeve can be rotated with respect to the fluid connecting piece and is pressure-tight to high pressures.

In general, the shape of the connecting sleeve 105 and the molded part 117 is not limited to the shape shown, so that, depending on the fluid sensor connecting piece, differently-adjusted forms can be used. Specifically, the connecting sleeve can be adjusted to the different fluid sensor connecting pieces by different molded parts.

All of the individual characteristics shown in the figures and explained in the description can be combined with one another in any arbitrary sensible manner in order to realize their advantageous effects simultaneously.

### REFERENCE LIST

100 Angle connector
101 Fluid sensor connecting piece
103 Line connection
105 Connecting sleeve
107 Longitudinal axis
109 Longitudinal axis
111 Inner fluid channel
113 Boss
115 Recess
117 Molded part
119 Sleeve inner wall
121 Retaining rib
123 Ribs
125 Reinforcing ribs
200 Fluid sensor system
201 Fluid sensor
203 Fluid line
205 Clamp

1. Angle connector for the connection of a fluid sensor connecting piece of a fluid sensor to a fluid line with:
a line connection for connecting of the fluid line; and
a connecting sleeve for holding of the fluid sensor connecting piece with positive locking.

2. Angle connector according to claim 1, wherein a longitudinal axis of the line connection and a longitudinal axis of the connecting sleeve are directed at an angle with respect to one another.

3. Angle connector according to claim 2, wherein the angle between the longitudinal axis of the line connection and the longitudinal axis of the connecting sleeve lies in an angle range of between 80° and 100°.

4. Angle connector according to claim 1, wherein the line connection and the connecting sleeve each have an inner fluid channel connected to one another with fluid technology.

5. Angle connector according to claim 1, wherein the connecting sleeve is designed to hold the fluid sensor connecting piece with a positive-locking connection.

6. Angle connector according to claim 1, wherein an inner wall of the connecting sleeve has at least one boss or recess for holding of the fluid sensor connecting piece with positive locking.

7. Angle connector according to claim 1, wherein in the connecting sleeve a molded part is arranged for holding of the fluid sensor connecting piece with positive locking.

8. Angle connector according to claim 7, wherein the molded part has a boss or a recess for holding of the fluid sensor connecting piece with positive locking.

9. Angle connector according to claim 7, wherein the molded part is removable or is permanently attached to an inner wall of the connecting sleeve.

10. Angle connector according to claim 7, wherein the molded part has at least two circumferential ribs for sealing the molded part against the connecting sleeve.

11. Fluid sensor system, with:
an angle connector according to claim 1;
a fluid sensor with a fluid sensor connecting piece, which is arranged in the connective sleeve and is connected to the connecting sleeve with positive locking; and
a fluid line, which is connected to the line connection.

12. Fluid sensor system according to claim 11, wherein a longitudinal axis of the line connection and a longitudinal axis of the connecting sleeve are directed at an angle with respect to one another.

13. Fluid sensor system according to claim 12, wherein the angle between the longitudinal axis of the line connection and the longitudinal axis of the connecting sleeve lies in an angle range of between 80° and 100°.

14. Fluid sensor system according to claim 11, wherein the line connection and the connecting sleeve each have an inner fluid channel connected to one another with fluid technology.

15. Fluid sensor system according to claim 11, wherein the connecting sleeve is designed to hold the fluid sensor connecting piece with a positive-locking connection.

16. Fluid sensor system according to claim 11, wherein an inner wall of the connecting sleeve has at least one boss or recess for holding of the fluid sensor connecting piece with positive locking.

17. Fluid sensor system according to claim 11, wherein in the connecting sleeve a molded part is arranged for holding of the fluid sensor connecting piece with positive locking.

18. Fluid sensor system according to claim 17, wherein the molded part has a boss or a recess for holding of the fluid sensor connecting piece with positive locking.

19. Fluid sensor system according to claim 17, wherein the molded part is removable or is permanently attached to an inner wall of the connecting sleeve.

20. Fluid sensor system according to claim 17, wherein the molded part has at least two circumferential ribs for sealing the molded part against the connecting sleeve.
